(19) United States
(12) Patent Application Publication
(10) Pub. No.: US 2011/0318304 A1
(43) Pub. Date: Dec. 29, 2011

(54) DETECTION OF EPHA3 AS A MARKER OF THE PRESENCE OF A SOLID TUMOR

(76) Inventors: Martin Lackmann, St. Andrews Beach (AU); Andrew Mark Scott, Kew East (AU); Catherine To, Bayswater (AU); Christopher R. Bebbington, South San Francisco, CA (US); Geoffrey T. Yarranton, South San Francisco, CA (US); Mark Baer, South San Francisco, CA (US); Varghese Palath, South San Francisco, CA (US)

(21) Appl. No.: 13/164,581

(22) Filed: Jun. 20, 2011

Related U.S. Application Data
(60) Provisional application No. 61/356,522, filed on Jun. 18, 2010.

(51) Int. Cl. A61K 38/20; G01N 33/574; A61P 35/00; A61K 39/395; A61K 38/21; C12Q 1/48; C12Q 1/68

(52) U.S. Cl. 424/85.2; 435/15; 435/7.23; 435/6.12; 424/174.1; 424/158.1; 424/85.4

(57) ABSTRACT

The invention provides methods and compositions for detecting non-hematopoietic, non-tumor EphA3-expressing cells in cancer patients and for monitoring the prognosis of patients using EphA3.

### CROSS-REFERENCE TO RELATED APPLICATIONS

This application claims benefit of U.S. provisional application 61/356,522, filed Jun. 18, 2010, which application is herein incorporated by reference.

### BACKGROUND OF THE INVENTION

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) control cell positioning and tissue organization (Poliakov, et al., Dev Cell 7:465-80, 2004). Among other patterning functions, various Ephs and ephrins have been shown to play a role in vascular development. Knockout of EphB4 and ephrin-B2 results in a lack of the ability to remodel capillary beds into blood vessels (Poliakov, et al., supra) and embryonic lethality. Persistent expression of some Eph receptors and ephrins has also been observed in newly-formed, adult micro-vessels (Brantley-Sieders, et al., Curr Pharm Des 10:3431-42, 2004; Adams, J Anat 202:105-12, 2003).

EphA3 is initially expressed—and guides—mesoderm patterning during gastrulation (see, e.g., Oates, et al., Mech Dev 83:77-94, 1999). EphA3−/− mice have a lethal cardiovascular phenotype. In agreement with its function in cardiovascular patterning, hypoxia-controlled expression of EphA3 on newly-emerging microvasculature, in particular on vascular and perivascular cells of solid tumors and in the regenerating human endometrium, an organ of cyclic adult neo-vascularisation was recently discovered. An agonistic anti-EphA3 antibody, by disrupting the tumor microvascular integrity, inhibits the growth of solid cancers, indicating a role for EphA3 in assembly and maintenance of tumor vessels (see, WO 2008/112192).

Tumor growth, invasiveness and metastasis involves an extensive dialogue between tumor-endothelial and stroma cells (Bhowmick et al., Nature 432:332-337, 2004). This tumor micro-environment develops initially by co-option and expansion of neighboring vascular and stromal tissues (e.g., Folkman, N Engl J Med 285:1182-1186, 1971), but also by active recruitment and subsequent proliferation of bone marrow-derived cells (BMDs), including circulating endothelial precursor cells (CEPs), mesenchymal stem cells (MSCs) and inflammatory leukocytes (e.g., Psaila & Lyden Nat Rev Cancer 9:285-293, 2009; Carmeliet, Nature 438:932-936, 2005). Low oxygen pressure, resulting from expansive tissue growth, stabilizes hypoxia inducible transcription factors (HIFs) (Pugh & Ratcliffe Nat Med 9:677-684, 2003), which induce expression of vasculogenic and angiogenenic proteins, in turn orchestrating recruitment and proliferation of vascular and mural cells and their assembly into new blood vessels. An important role of these CD133⁺ (human) CEPs, PDGFRβ⁺/αSMA⁺ perivascular progenitor cells, infiltrating leukocytes (Lyden, Nat Med 7: 1194-1201, 2001) (tumor-associated macrophages, VEGFR1⁺ myeloid progenitors) and MSCs (McAllister et al., Cell 133:994-1005, 2008) in tumor progression and metastasis has been confirmed in a number of recent preclinical and clinical studies (Psaila, 2009, supra). There is now evidence suggesting that angiogenic and pro-inflammatory cytokines recruit BMD myeloid and MSCs globally, and mark out pre-metastatic sites already prior to the arrival of tumor cells (McAllister, 2008, supra; Karnoub, et al. Nature 449:557-563, 2007).

Not surprisingly, the role of BMD precursor cells created great interest for their use as surrogate markers monitoring pathologies such as heart disease and cancer (Carmeliet, supra, Rafii et al., Nat Med 9:702-712, 2003). To date, enumeration of CEPs by multicolor flow cytometry has been used to stage and stratify patients with glioblastoma (Batchelor, et al. Cancer Cell 11:83-95, 2007), Non-Small Cell Lung Cancer (Dome, et al., Cancer Res 66: 7341-7347, 2006) and rectal cancer (Duda, et al., J Clin Oncol 24:1449-1453, 2006), and was used to predict the clinical benefit of “metronomic” chemotherapy for cancer (Mancuso, et al., Blood 108:452-459, 2006). However, this analysis relies on a combination of cell surface markers, including CD31, CD34, CD45, CD105, CD133, CD146, CD309 (KDR) to distinguish viable circulating endothelial cells (CECs) and CEPs. Detailed protocols have been proposed to standardize the phenotypic definition of CEPs (Duda, et al., Nature protocols 2; 805-810, 2007; Yoder, et al., Blood 109; 1801-1809, 2007; Mancuso, et al., Clin Cancer Res 15:267-273, 2009), but substantial difficulties inherent to multiparameter analysis for the distinction between CECs, CEPs and hematopoietic cells have limited the routine use of this assay.

Among the bone marrow-derived progenitor cell populations, MSCs constitute a rare non-hematopoietic pluripotent, colony-forming units (CFU)-fibroblastic cell population, which are characterized by their ability in vitro and in vivo to differentiate into mesodermal, endothelial, ectodermal and endodermal lineages (Jiang, et al., Proc Natl Acad Sci USA 101:16891-16896, 2004; Jiang, et al., Nature 418:41-49, 2002). Their complex functional and gene expression profile and multilineage differentiation potential (Pittenger, et al., Science 284:143-147, 1999) has lead to some confusion about their classification as conventional ‘stem cells’, and the term ‘BM-derived multipotent mesenchymal stromal cell’ (BM-MSC) has been proposed to designate these plastic-adherent, mesenchymal progenitor cells (Horwitz, et al., Cytotherapy 7: 393-395, 2005). While specific markers for their definition are lacking, a range of different cell surface proteins, including CD44, CD73, CD90, CD105, and CD309 (KDR) and in particular the lack of hematopoietic (CD34, CD45, CD14) and endothelial markers (CD34, CD31, vWF) are commonly used for their identification (Jiang et al., supra 2002; Roorda, et al., Crit Rev Oncol Hematol 69:187-198, 2009). The contribution of BM-MSC's to tissue assembly are apparent only during tissue remodeling after injury or inflammation; considerable evidence confirmed their important contribution to tumor growth and metastasis, in particular their participation as tumor fibroblasts in tumor stroma, as endothelial- and pericyte-like cells in tumor vasculature, and their capacity to instigate metastasis at secondary sites (references cited above). In accordance with their central role in pathological tissue remodeling, circulating peripheral blood MSCs have been identified and described in a number of species, although at very much reduced frequency compared to the BM-derived counterparts, in particular in human peripheral blood samples (He, et al., Stem Cells 25:69-77, 2007). Nevertheless, levels of MSCs were found elevated in peripheral blood of breast cancer patients (Tondreau, et al., Stem Cells 23:1105-1112, 2005), and the similarity of cell surface marker expression profile and multi-lineage potential (He, 2007, supra; Ahn & Brown, Angiogenesis 12:159-164, 2009) confirms the BM origin and role in tumor progression of these circulating MSCs, suggested from bone marrow transplantation experiments.

The essential role of bone marrow-derived endothelial, stromal and haematopoietic progenitor cells during pathological tissue re-modelling and oncogenic growth and their increased presence in the circulation of patients, has generated great interest for their use as surrogate diagnostic markers in a range of diseases, including cancer. However, difficulties in their accurate phenotypic characterization have presented difficulties in developing diagnostic and prognostics. The present invention overcomes these problems.

### BRIEF SUMMARY OF THE INVENTION

The present invention is based, in part, on the discovery of EphA3⁺ non-hematopoietic, non-tumor cells in human peripheral blood samples, whereby the frequency of these cells is significantly increased in solid tumor cancer patients. The level of these cells drops following anti-vascular cancer therapy, thus serving as an indicator of therapeutic efficacy. In some embodiments, the level of EphA3+ non-hematopoietic, non-tumor cells in the peripheral blood may be used to diagnose the presence of a tumor. Accordingly, the invention provides a method of screening for the presence of EphA3+ non-hematopoietic, non-tumor cells in peripheral blood of a patient that has a solid tumor, or is suspected of having a tumor.

In one aspect, the invention provides a method of identifying a population of non-hematopoietic, non-tumor EphA3⁺ cells in a biological sample from a patient that has a solid tumor, or that may have a solid tumor, the method comprising: providing a sample comprising peripheral blood cells from a patient that has the solid tumor, or that may have a solid tumor; and detecting expression of EphA3+ in non-hematopoietic, non-tumor cells in the sample. An increase in the level of such cells above normal is indicative of the presence of a tumor. The methods of the invention can further comprise detecting other cell surface markers in addition to EphA3. These surface markers include, CD34, CD45, CD44, CD90, and/or KDR. Typically, EphA3⁺ cells from non-hematopoietic, non-tumor cells are CD34⁻ and CD45⁻, but express CD44, CD90 and KDR. In some embodiments, the methods comprise detecting expression of EphA3 and determining whether the EphA3⁺ cells are CD34⁻ and/or CD45−. In some embodiments, the methods further comprise determining whether the EphA3⁺ cells express CD44, CD90, and/or KDR. In some embodiments, the methods further comprise determining whether the EphA3+ cells express CD34, CD45, CD44, CD90, and KDR.

In some embodiments, the step of detecting expression of EphA3 comprises detecting expression on the surface of non-hematopoietic, non-tumor cells. Typically, the step of detecting expression on the surface of the non-hematopoietic, non-tumor cells comprises contacting the cells with a first antibody that selectively binds to EphA3. In some embodiments, flow cytometry is used. In some embodiments, the method can further comprise contacting the non-hematopoietic, non-tumor cells with a second antibody that binds to EphA3 at a different epitope. In typical embodiments, the second antibody does not compete with the first antibody for binding to EphA3. The first and second antibody may be labeled with the same detectable label. Alternatively, the first and the second antibodies may be labeled with different detectable labels.

In some embodiments, the methods of the invention are employed using a sample that comprise peripheral blood cells where the sample is from a patient that has a breast carcinoma, a lung adenocarcinoma, a lung squamous cell carcinoma, a colon adenocarcinoma, a renal cell carcinoma, a transitional cell carcinoma, a prostate adenocarcinoma, a melanoma, or a glioblastoma. In some embodiments, the methods of the invention are employed using a sample from a patient that is suspected of having a solid tumor, e.g., a breast carcinoma, a lung adenocarcinoma, a lung squamous cell carcinoma, a colon adenocarcinoma, a renal cell carcinoma, a transitional cell carcinoma, a prostate adenocarcinoma, a melanoma, or a glioblastoma.

In some embodiments, the step of detecting expression of EphA3 comprises an RT-PCR reaction. The RT-PCR reaction may be performed on single cells or on populations of cells that have been sorted to remove hematopoietic cells.

Often, the methods of the invention may further comprise administering a cancer therapeutic agent to the patient. In some embodiments, the agent is administered when a patient has a level of EphA3-expressing non-hematopoietic, non-tumor cells in the blood that is greater than 0.01%, typically greater than 0.02%, and often greater than 0.025% or 0.05%. The cancer therapeutic agent may be any agent used to treat a tumor. These include agents that are anti-vascular agents, such as vascular endothelial growth factor (VEGF) antagonists or antibodies that activate EphA3, leading to cell rounding. In other embodiments, the cancer therapeutic agent may be an antibody that selectively binds to EphA3 and is cytotoxic to the cells via ADCC and/or phosphorylates EphA3 and causes apoptosis.

In a further aspect, the invention provides a method of monitoring efficacy of a cancer therapeutic agent, the method comprising: determining the level of EphA3+ non-hematopoietic, non-tumor cells in peripheral blood from a patient that has a solid tumor following a treatment with the cancer therapeutic agent; and comparing the level of EphA3+ non-hematopoietic, non-tumor cells in peripheral blood to the level prior to the treatment with the cancer therapeutic agent. In some embodiments the cancer therapeutic agents is an anti-vascular-therapeutic agent, such as a VEGF antagonist or an antibody that activates EphA3 and induces cell rounding. In some embodiments, the cancer therapeutic agents is an antibody that selectively binds EphA3 and is cytotoxic via ADCC and/or activates EphA3 and causes apoptosis.

In some embodiments, a method of the invention for monitoring therapeutic efficacy may further comprise determining whether the EphA3+ cells are CD34− and/or CD45⁻. In some embodiments, the method may further comprise detecting expression of CD44, CD90 and KDR. In typical embodiments, non-hematopoietic, non-tumor cells that are monitored to evaluate therapeutic efficacy of a cancer agent are EphA3+, CD34−, CD45−, CD44+, CD90+ and KDR+, although other cell surface markers may be present.

In typical embodiments, the method of determining the level of EphA3+ non-hematopoietic, non-tumor cells to monitor therapeutic efficacy comprises detecting EphA3 expression on the surface of the non-hematopoietic, non-tumor cells, preferably by detecting expression on the surface of the cells comprising contacting the cells with a first antibody that selectively binds to EphA3. In some embodiments, flow cytometry is used to detect EphA3 on the surface of non-hematopoietic, non-tumor cells. In some embodiments, the method further comprises contacting the cells with a second antibody that selectively binds to a different EphA3 epitope. In some embodiments, the second antibody does not compete with the first antibody for binding to EphA3. The first and second antibody may be labeled with the same detectable labels; or different detectable labels may be employed.

In alternative embodiments, the method of monitoring therapeutic efficacy of a cancer therapeutic agent may comprise detecting expression of EphA3+ on non-hematopoietic, non-tumor cells in the peripheral blood using an amplification reaction such as RT-PCR. In some embodiments, the RT-PCR reaction is performed on peripheral blood cells from a patient from where the hematopoietic cells have been removed from the sample.

In some embodiments, therapeutic efficacy of a cancer agent is monitored in accordance with the invention for a patient that has a breast carcinoma, a lung adenocarcinoma, a lung squamous cell carcinoma, a colon adenocarcinoma, a transitional cell carcinoma, a renal cell carcinoma, a prostate adenocarcinoma, a melanoma, or a glioblastoma.

In a further aspect, the invention provides a kit for detecting the presence of EphA3⁺ non-hematopoietic cells, non-tumor in a sample, the kit comprising a first antibody that selectively binds to an EphA3 epitope and a second antibody that selectively binds to a different EphA3 epitope. The first and second antibodies may be labeled. In some embodiments, the first and second antibodies are labeled with the same detectable label whereas in alternative embodiments, the first and second antibodies are labeled with different labels. In some embodiments, the kit may further comprise one or more of the following antibodies: an antibody that selectively binds to CD34, an antibody that selectively binds to CD45, an antibody that selectively binds to CD44, an antibody that selectively binds to CD90, and/or an antibody that selectively binds to KDR.

In some embodiments, the detection methods of the invention may be used to detect the presence of a solid tumor in a patient that is suspected of having cancer, e.g., a patient that has a symptom of cancer.

### BRIEF DESCRIPTION OF THE DRAWINGS

FIG. 1 provides data showing EphA3 expression patterns on tumor vasculature and stroma.

FIGS. 2a and 2b provide data showing that EphA3 is expressed on a population in human peripheral blood distinct from circulating endothelial progenitor cells.

FIGS. 3a and 3b provides data showing flow cytometric quantitation of CEP and EphA3⁺ cells in peripheral blood.

FIGS. 4a and 4b provides data showing the concentration of CEPs and of EphA3⁺ mural cells in peripheral blood of cancer patients.

FIGS. 5a and 5b provides data showing the concentration of CEP's and EphA3− following anti-vascular therapy.

FIG. 6 provides data from an ELISA demonstrating binding of anti-EphA3 antibodies to EphA3-EphrinA5 complex. Antibodies were bound to either EphA3 or pre-formed EphA3-EphrinA5 complexes. Detection of antibodies was with goat anti-mouse HRP conjugate revealed using TMB. Broken lines indicate binding to EphA3-EphrinA5 complexes and solid lines indicate binding to EphA3. Data was analyzed by Prism 5.0 software.

FIG. 7A-7D provide data showing binding of purified anti-EphA3 antibodies to LK63 cells analyzed by flow cytometry. Binding of each antibody (25 nM) is revealed using FITC-conjugated anti-mouse antibody. Background binding of the conjugated anti-mouse secondary antibody alone is shown as the filled histogram.

FIG. 8 provides data showing binding of Alexa488-conjugated SL-2 to primary bone marrow cells from an AML patient determined by flow cytometry.

### DETAILED DESCRIPTION OF THE INVENTION

### Definitions

As used herein “solid tumor” refers to an abnormal mass of tissue comprising neoplastic cells in a subject. Solid tumors may be benign or malignant. Solid tumors that can be detected and/or monitored using the methods and compositions of the invention are characterized by neovascularization. The tumor vasculature (also referred to as microvasculature) is characterized by rapid proliferation of the endothelial cells, poor wall structure, increased permeability to plasma proteins, and a limited ability to increase blood flow in response to demand. The tumor vasculature allows the tumor cells of the tumor mass to acquire a growth advantage compared to the normal cells. Solid tumors are named for the type of cells that form them. Examples of solid tumors are sarcomas, carcinomas (epithelial tumors), melanomas, and glioblastomas.

The terms “cancer cell” or “tumor cell” are used interchangeably to refer to a neoplastic cell. The term includes cells that are benign as well as malignant. Neoplastic transformation is associated with phenotypic changes of the tumor cell relative to the cell type from which it is derived. The changes can include loss of contact inhibition, morphological changes, and aberrant growth. (see, Freshney, Culture of Animal Cells a Manual of Basic Technique (3^{rd} edition, 1994).

In the context of this invention, a “non-hematopoietic, non-tumor cell” refers to a cell that is not of hematopoietic origin and is not neoplastic. Such a cell may be present in a tumor mass or a site such as the blood stream. Non-hematopoietic, non-tumor cells include EphA3+ stromal progenitor cells.

As used herein, an “EphA3⁺ stromal progenitor cell” is used interchangeably with the term “mesenchymal EphA3⁺ cell” or “CD34⁻EphA3⁺ progenitor cell” to refer to a cell that is characteristic of a population of cells found in the peripheral blood of cancer patients that has the phenotype CD34⁻CD45⁻CD44⁺CD90⁺KDR⁺EphA3⁺, (other cell surface markers may also be present on this cell population). These cells are elevated in cancer patients compared to normal patients (i.e., individuals that do not have cancer). As known in the art, these surface antigens may have alternative names. For example, KDR⁺ cells may also be referred to as CD309⁺ cells or VEGFR-2⁺ cells.

As used herein, the term “cells from a solid tumor” refer to tumor cells or cells that are present in a tumor that are not neoplastic. These include cells such as vascular cells, including endothelial cells and smooth muscle cells.

“Inhibiting growth of a cancer” in the context of the invention refers to slowing growth and/or reducing the cancer cell burden of a patient that has cancer. “Inhibiting growth of a cancer” thus includes killing cancer cells.

As used herein, the terms “cancer therapeutic agent” or “anti-cancer therapeutic agent” are used interchangeably to refer to an agent that when administered to a patient suffering from cancer, in a therapeutically effective dose, will cure, or at least partially arrest the symptoms of the disease and complications associated with the disease.

As used herein, “tumor vasculature endothelial cells” are endothelial cells that are present in the vasculature of a tumor or precursor cells that become part of the vasculature of a tumor.

An “anti-vascular therapeutic agent” or “anti-vasculogenic therapeutic agent” as used herein refers to a treatment that inhibits vasculogenesis and blood vessel formation.

Examples of such agents include anti-EphA3 antibodies and vascular endothelial growth factor (VEGF) antagonists.

As used herein “EphA3” refers to the Eph receptor A3. This receptor has also been referred to as “Human embryo kinase”, “hek”, “eph-like tyrosine kinase 1”, “etk1” or “tyro4”. EphA3 belongs to the ephrin receptor subfamily of the protein-tyrosine kinase family. Eph and Eph-related receptors have been implicated in mediating developmental events. Receptors in the Eph subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands. EphA3 binds ephrin-A ligands. EphA3 nucleic acid and protein sequences are known. An exemplary human EphA3 amino acid sequence is available under accession number (EAW68857).

In the present invention, “EphA3 antibody” or “anti-EphA3 antibody” are used interchangeably to refer to an antibody that specifically binds to EphA3.

In the context of this invention, and antibody that “activates” EphA3 causes phosphorylation of EphA3 and typically, rounding of the cell.

The term “mAb IIIA4” refers to monoclonal antibody IIIA4 that was originally raised against LK63 human acute pre-B leukemia cells to affinity isolate EphA3 (Boyd, et al. J Biol Chem 267:3262-3267, 1992). mAb IIIA4 binds to the native EphA3 globular ephrin-binding domain (e.g., Smith, et al., J. Biol. Chem 279:9522-9531, 2004). It is deposited in the European Collection of Animal Cell Cultures under accession no. 91061920 (see, e.g., EP patent no. EP0590030).

A VEGF antagonist as used herein refers to an agent that inhibits VEGF-mediated signaling, such as antibodies that bind to VEGF and inhibit VEGF receptor binding or an antibody that binds to a VEGF receptor and inhibits VEGF binding.

As used herein, an “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” as used herein includes antibody fragments that retain binding specificity. For example, there are a number of well characterized antibody fragments. Thus, for example, pepsin digests an antibody C-terminal to the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the invention also include heavy chain dimers, such as antibodies from camelids. For the purposes of this invention, antibodies are employed in a form that can bind to EphA3 present on the surface of stromal progenitor cells.

As used herein, “V-region” refers to an antibody variable region domain comprising the segments of Framework 1, CDR1, Framework 2, CDR2, and Framework3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

“Epitope” or “antigenic determinant” refers to a site on an antigen to which an antibody binds. Epitopes can be foamed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in which CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci. USA 91: 969, 1994).

A “Humaneered™” antibody in the context of this invention refers to an engineered human antibody having a binding specificity of a reference antibody. A “Humaneered™” antibody for use in this invention has an immunoglobulin molecule that contains minimal sequence derived from a donor immunoglobulin. Typically, an antibody is “Humaneered™” by joining a DNA sequence encoding a binding specificity determinant (BSD) from the CDR3 region of the heavy chain of the reference antibody to human V_{H} segment sequence and a light chain CDR3 BSD from the reference antibody to a human V_{L} segment sequence. Methods for humaneering are provided in US patent application publication no. 20050255552 and US patent application publication no. 20060134098.

A “human” antibody as used herein encompasses humanized and Humaneered™ antibodies, as well as human monoclonal antibodies that are produced using known techniques.

A “therapeutic” antibody as used herein refers to a human or chimeric antibody that is administered to a patient that has a solid tumor.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The phrase “specifically (or selectively) binds” to an antibody or “specifically (or selectively) immunoreactive with,” when referring to a protein or peptide, refers to a binding reaction where the antibody binds to the protein of interest. In the context of this invention, the antibody typically binds to EphA3 with an affinity that is at least 100-fold better than its affinity for other antigens.

The term “equilibrium dissociation constant (K_{D}) refers to the dissociation rate constant (k_{d}, time⁻¹) divided by the association rate constant (kₐ, time⁻¹, M⁻¹). Equilibrium dissociation constants can be measured using any known method in the art. The antibodies of the present invention are high affinity antibodies. Such antibodies have an affinity better than 500 nM, and often better than 50 nM or 10 nM. Thus, in some embodiments, the antibodies of the invention have an affinity in the range of 500 nM to 100 pM, or in the range of 50 or 25 nM to 100 pM, or in the range of 50 or 25 nM to 50 pM, or in the range of 50 nM or 25 nM to 1 pM.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), radioactive labels, biotin, digoxigenin, or haptens and proteins or other entities which can be made detectable. The labels may be incorporated into the antibodies at any position. Moreover the labels need not be directly conjugated to the anti-EphA3 antibody, but can be present on a secondary detection agents, such as a secondary antibody that binds the anti-EphA3 antibody. Any method known in the art for conjugating the antibody to the label may be employed, e.g., using methods described in Hermanson, Bioconjugate Techniques 1996, Academic Press, Inc., San Diego.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “a” or “an” is generally intended to mean “one or more” unless otherwise indicated.

### Introduction

The invention is based, in part, on the discovery that EphA3-expressing non-hematopoietic, non-tumor cells in the peripheral blood of cancer patients that have a solid tumor. Such cells are reduced following treatment with a cancer therapeutic agent, e.g., an anti-vasculogenic therapy, and can be used as an indicator of therapeutic efficacy of a treatment in such patients. Further, such cells can be used to diagnose the presence of a solid tumor in a patient, for example, a patient that has a symptom of cancer, or is otherwise suspected of having cancer.

A patient that can be monitored, or diagnosed, in accordance with the invention includes patients having any kind of solid tumor, or suspected of having any kind of solid tumor, where it is desirable to administer a cancer agent, such as an anti-EphA3 antibody. These tumors include solid tumors such as, including breast carcinomas, lung carcinomas, prostate carcinomas, gastric carcinomas, esophageal carcinomas, colorectal carcinomas, liver carcinomas, ovarian carcinomas, vulval carcinomas, kidney carcinomas, transitional cell carcinomas, cervical carcinomas, endometrial carcinoma, endometrial hyperplasia, endometriosis, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreatic carcinomas, retinoblastoma, astrocytoma, glioblastoma, Schwannoma, oligodendroglioma, medulloblastoma, neuroblastomas, sarcomas include fibrosarcomas, rhabdomyosarcoma, osteogenic sarcoma, leiomyosarcomas, urinary tract carcinomas, thyroid carcinomas, Wilm's tumor, brain tumors, and renal cell carcinomas. Patients that may be monitored or diagnosed in accordance with the invention may also have abnormal vascular proliferation associated with phakomatoses, and edema (such as that associated with brain tumors).

Identification of Patients that have EphA3⁺ Non-Hematopoietic, Non-Tumor Cells

Cancer patients that have EphA3+ non-hematopoietic, non-tumor cells can be identified by detecting the expression of EphA3 on peripheral blood cells from the patient.

EphA3 expression can be detected using methods well known in the art. Often, an immunological assay can be used to detect the presence of EphA3 protein on the surface of the cells. Immunological assays include ELISA, fluorescent-activated cell sorting, and the like. Alternatively EphA3 expression can be detected by detecting the level of mRNA encoding EphA3. For example, a nucleic acid amplification method, e.g., an RT-PCR is employed to quantify the amount of RNA.

In order to identify the cells as EphA3+ non-hematopoietic, non-tumor cells, the cells are typically also evaluated for expression of CD45. Optionally, the cells can further be evaluated for one or more of CD34, CD44, CD90, and KDR.

A sample comprising peripheral blood cells, is obtained from the patient for evaluating EphA3 expression. The blood cells are evaluated for the expression of EphA3, and optionally, CD34 or other markers, using known techniques, e.g., flow cytometry or other antibody-based assays.

A patient is considered positive for EphA3⁺ non-hematopoietic, non-tumor cells if the level of EphA3+ mononuclear cells in peripheral blood is greater, e.g., at least 2-fold, and preferably at least 5- or 10-fold, than the level observed in normal individuals who do not have a solid tumor. In normal individuals, typically only about 0.002% of peripheral blood mononuclear cells are EphA3+. Thus, a level above normal values is indicative of the presence of a solid tumor.

Cancer patients that have EphA3+ non-hematopoietic, non-tumor cells are typically treated with a cancer therapeutic agent that targets solid tumors. Such agents include anti-vascular therapeutic agents, including an anti-EphA3 antibody that activate EphA3; and/or an agents such as a VEGF antagonist. Examples of anti-EphA3 antibodies that target vasculature are described, e.g., in WO 2008/112192. Other examples of anti-EphA3 antibodies that can be used for treatment of patients that have a solid tumor are provided in WO/2011/053465.

In some embodiments, the invention provides methods of monitoring the therapeutic efficacy of a cancer treatment for a patient that has a solid tumor. Following treatment with a therapeutic agent, a peripheral blood sample from the patient may be obtained and evaluated to determine whether the level of EphA3+ non-hematopoietic, non-tumor cells has decreased relative to the level before treatment. A decrease in the concentration of such cells by at least 20%, typically at least 50% or 100% or more, is indicative of a therapeutic effect of the treatment.

In some embodiments, the presence of EphA3+ non-hematopoietic, non-tumor cells in a patient is indicative that the patient will be responsive to other cancer therapeutic agents that inhibit cell growth. Such compounds may or may not cause cell death. Cytotoxic agents that can be administered to a patient that has EphA3+ stromal cells include compounds such as antibodies, e.g., VEGF antagonists, Her2/neu antibodies; agents such as L-asparaginase, interleukins, interferons, aromatase inhibitors, antiestrogens, anti-androgens, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, protein or lipid phosphatase targeting agents, anti-angiogenic agents, anti-apoptotic pathway inhibitors, apoptotic pathway agonists, telomerase inhibitors, protease inhibitors, metalloproteinase inhibitors, and aminopeptidase inhibitors. Examples of such agents include, but are not limited to, cisplatin, cyclophosphamide, dacarbazine, ifosfamide, mechlorethamine, melphalan, carmustine, estramutine, lomustine, 5-fluorouracil, methotrexate, genistein, taxol, gemcitabine, cytarabine, fludarabine, busulfan, bleomycin, dactinomycin, daunorubicin, doxorubicin, idarubicin, epirubicin, esorubicin, detorubicin, taxanes such as paclitaxel and docetaxel, etoposide, vinca alkaloids such as vinblastine and vincristine, vinorelbine, amsacrine, tretinoin, dacarbazine (DTIC), actinomycins, maytansinol, rifamycin, streptovaricin, caminomycin, mitoxantrone, bleomycins, mitomycins, camptothecins, bortezomib, temozolomide, combretastatin, combretastatin A-2, combretastatin A-4, calicheamicins, leuprolide, and pegaspargase, fluorodeoxyuridine, ptorafur, 5′-deoxyfluorouridine, capecitabine, tamoxifen, toremefine, tolmudex, thymitaq, flutamide, fluoxymesterone, bicalutamide, finasteride, trioxifene, leuproelin acetate, estramustine, droloxifene, megesterol acetate, aminoglutethimide, testolactone, mitomycins A, B and C, mithramycin, anthramycin, porfiromycin, carboplatin, oxaliplatin, tetraplatin, platinum-DACH, ormaplatin, thalidomide, lenalidomide, telomestatin, podophyllotoxin, epipodophyllotoxin, teniposide, aminopterin, methopterin, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, leurosine, leurosidine, vindesine, ethylenimines and methylmelamines.

### Anti EphA3 Antibodies

Any number of commonly used immunoassays can be used to detect EphA3 expression. A general overview of the applicable antibody technology can be found in Harlow & Lane, Antibodies: A Laboratory Manual (1988) and Harlow & Lane, Using Antibodies (1999). Other resources include see also Methods in Cell Biology: Antibodies in Cell Biology, volume 37 (Asai, ed. 1993); Basic and Clinical Immunology (Stites & Terr, eds., 7th ed. 1991, and Current Protocols in Immunology (Coligan, et al. Eds, John C. Wiley, 1999-present). Immunological binding assays can use either polyclonal or monoclonal antibodies.

Commonly used assays include noncompetitive assays (e.g., sandwich assays) and competitive assays. Commonly used assay formats include flow cytometry-based assay as well as other immunoassays.

The anti-EphA3 antibodies to detect EphA3 on the surface of cells can be raised against EphA3 proteins, or fragments, or produced recombinantly. Any number of techniques can be used to determine antibody binding specificity. See, e.g., Harlow & Lane, supra, for a description of immunoassay formats and conditions that can be used to determine specific immunoreactivity of an antibody.

In some embodiments, the anti-EphA3 antibody is a polyclonal antibody. Methods of preparing polyclonal antibodies are known to the skilled artisan (e.g., Harlow & Lane, Methods in Immunology, both supra). Polyclonal antibodies can be raised in a mammal by one or more injections of an immunizing agent and, if desired, an adjuvant. The immunizing agent includes an EphA3 receptor protein, or fragment thereof.

In some embodiments, the anti-EphA3 antibody is a monoclonal antibody. Monoclonal antibodies may be prepared using hybridoma methods. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

In some embodiments, it may be desirable to use human monoclonal antibodies. Human monoclonal antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

### Antibody Specificity

One example of an antibody suitable for use with the present invention is an antibody that has the binding specificity of mAb IIIA4. The monoclonal antibody mAb IIIA4 binds to the native EphA3 globular ephrin-binding domain (Smith et al., J. Biol. Chem. 279:9522-9531, 2004; and Vearing et al., Cancer Res. 65:6745-6754, 2005). High affinity mAb IIIA4 binding to the EphA3 surface has little effect on the overall affinity of ephrin-A5 interactions with EphA3.

Examples of human engineered antibodies that are suitable for use with the present invention, e.g., as therapeutic antibodies administered to a patient determined to have a level of EphA3+ non-hematopoietic, non-tumor cells in peripheral blood above normal, are provided in WO/2011/053465. Such antibodies may also be used diagnostically.

If an anti-EphA3 antibody is used therapeutically, subsequent analysis of the level of EphA3+ non-hematopoietic, non-tumor cells in peripheral blood cells to monitor therapeutic efficacy preferably employs an antibody that binds to a different epitope of EphA3.

In some embodiments, a monoclonal antibody that competes with mAb IIIA4 for binding to EphA3, or that binds the same epitope as mAb IIIA4, is used to assess EphA3 expression on peripheral blood cells. Any of a number of competitive binding assays can be used to measure competition between two antibodies for binding to the same antigen. For example, a sandwich ELISA assay can be used for this purpose. In an exemplary assay, ELISA is carried out by using a capture antibody to coat the surface of a well. A subsaturating concentration of tagged-antigen is then added to the capture surface. This protein will be bound to the antibody through a specific antibody:antigen interaction. After washing, a second antibody that is linked to a detectable moiety is added to the ELISA. If this antibody binds to the same site on the antigen as the capture antibody, or interferes with binding to that site, it will be unable to bind to the target protein as that site will no longer be available for binding. If however this second antibody recognizes a different site on the antigen it will be able to bind. Binding can be detected by quantifying the amount of detectable label that is bound. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine specificity. The ability of a particular antibody to recognize the same epitope as another antibody is typically determined by such competition assays.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

In some embodiments, the antibody binds to the same epitope as mAb IIIA4. The epitope for IIIA4 and human engineered derivatives resides in the N-terminal globular ligand binding domain of EphA3 (amino acids 29-202 in the partial human EphA3 sequence below):

| | |
|---|---|
| 1 | MDCQLSILLL LSCSVLDSFG ELIPQPSNEV NLLDSKTIQG ELGWISYPSH GWEEISGVDE |
| 61 | HYTPIRTYQV CNVMDHSQNN WLRTNWVPRN SAQKIYVELK FTLRDCNSIP LVLGTCKETF |
| 121 | NLYYMESDDD HGVKFREHQF TKIDTIAADE SFTQMDLGDR ILKLNTEIRE VGPVNKKGFY |
| 181 | LAFQDVGACV ALVSVRVYFK KC |

The IIIA4 antibody binds adjacent to but does not interfere substantially with binding of EphrinA5 to the receptor. The epitope for antibody IIIA4 has been further characterized by Smith et al., J. Biol. Chem. 279: 9522, 2004 using site-directed mutagenesis. In this analysis, mutation of Glycine at position 132 to Glutamic acid (G132E) abolishes binding to IIIA4. Mutation of Valine 133 to Glutamic acid (V133E) reduces binding of EphA3 to IIIA4 antibody approximately 100-fold. It has subsequently been observed by the inventors that Arginine 136 is also part of the epitope. This residue is changed to Leucine in the sequence of the highly conserved EphA3 protein in the rat (R136L). Rat EphA3 does not bind IIIA4 or a human engineered derivative of IIIA4. Thus, G132, V133 and R136 (bolded and underlined in the sequence above) are important amino acids within the IIIA4 epitope.

In some embodiments, EphA3+ cells can be detected using a second antibody to an epitope that is different from the IIIA4 antibody epitope. Thus, in some embodiments, a second EphA3 antibody is employed that does not compete with binding with the first EphA3 antibody.

### Binding Affinity

In some embodiments, the antibodies suitable for use with the present invention have a high affinity binding for human EphA3. For the purposes of this invention, high affinity binding between an antibody and an antigen exists if the dissociation constant (K_{D}) of the antibody is <about 10 nM, for example, about 5 nM, or about 2 nM, or about 1 nM, or less. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BIAcore™ 2000 instrument (Biacore AB, Freiburg, Germany) using CM5 sensor chips, as described by Krinner et al., (2007) Mol. Immunol. February; 44(5):916-25. (Epub 2006 May 11)).

The anti-EphA3 antibody for detection of EphA3 expression can bind to any region of EphA3.

The antibodies used to detect EphA3 expression are typically labeled with a detectable label. The particular label or detectable group used in the assay is not a critical aspect of the invention, as long as it does not significantly interfere with the specific binding of the antibody used in the assay. The label can be directly attached to the antibody or to another agents used in the detection assay, such as a secondary antibody. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include fluorescent compounds (e.g., fluorescein isothiocyanate, Texas red, rhodamine, fluorescein, Alexafluor 488, Alexafluor 647 and the like), radiolabels, enzymes (e.g., horse radish peroxidase, alkaline phosphatase and others commonly used in an ELISA), streptavidin/biotin, and colorimetric labels such as colloidal gold or colored glass or plastic beads (e.g., polystyrene, polypropylene, latex, etc.), or magnetic beads including magnetic microbeads. The antibody may be directly conjugated to the beads. Alternatively, the beads may be conjugated to a reagent capable of capturing the antibody, such as streptavidin-conjugated magnetic microbeads which are capable of binding biotinylated antibodies. In some embodiments, an antibody-capture bead for use in the invention is a streptavidin-conjugated microbead conjugated to a fluorescent label such as Alexafluor 647. Chemiluminescent compounds may also be used.

In some embodiments in which two antibodies are employed to detect EphA3 expression on non-hematopoietic, non-tumor cells present in the blood of patients that have a solid tumor, or are suspected of having a solid tumor, the two antibodies may be labeled with the same label to amplify the signal. In other embodiments, the antibodies may be labeled with different labels.

### RNA Expression

In other embodiments, EphA3 expression can be detected by detecting the level of mRNA encoding EphA3 that is expressed in peripheral blood cells. Often, a nucleic acid amplification method, e.g., an RT-PCR is employed to quantify the amount of RNA. In such embodiments, expression of other surface markers, CD34, CD45, CD44, CD90, KDR, is also typically evaluated. In such embodiments, an amplification method may be employed on a population of cells that is first sorted, e.g., by flow cytometry, to determine if it expresses a surface marker such as CD34.

The following examples are provided by way of illustration only and not by way of limitation. Those of skill in the art will readily recognize a variety of non-critical parameters that could be changed or modified to yield essentially similar results.

### Kits

The invention additionally provides kits for assessing EphA3 expression on non-hematopoietic cells, non-tumor cells in the blood of patients that have a solid tumor, or are suspected of having a solid tumor. A kit can comprise one or more antibodies that selectively bind EphA3. In typical embodiments, a kit of the invention comprises two antibodies that bind to EphA3 at different epitopes. In some embodiments, the two antibodies are labeled with the same detectable label. Alternatively, the two antibodies may be labeled with different detectable labels.

A kit can optionally include other components, such as antibodies to other surface markers such as CD34, CD45, CD44, CD90, or KDR; or buffers, materials, and components that can be used to detect EphA3 expression.

### EXAMPLES

### Example 1

### EphA3 Expression Patterns on Tumor Vasculature and Stroma

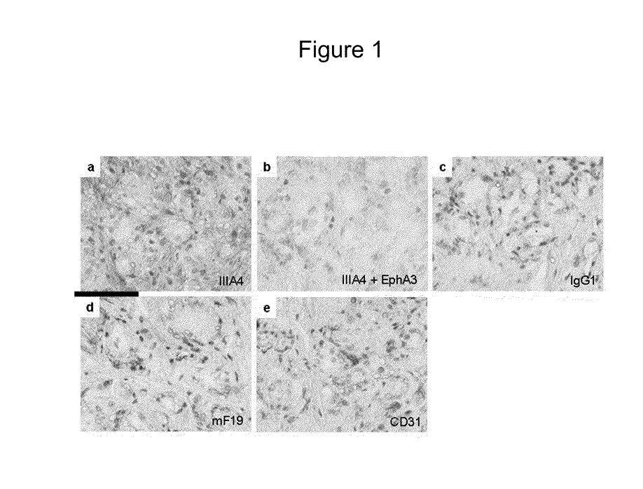

As an example to illustrate tumor stromal expression, the mouse IIIA4 antibody was used to analyze frozen tumor sections of human prostate carcinoma (acinar adenocarcinoma, n=8) by immuno-histochemistry (FIG. 1a). The specificity of the IIIA4 staining pattern was confirmed in parallel sections (FIG. 1b) where the IIIA4 mAb was used in the presence 60× molar excess of recombinant soluble EphA3 extracellular domain, reducing the anti-EphA3 signal to background level observed in control sections tested with a secondary antibody only (FIG. 1c). Further sections were analyzed with anti-fibroblast activating protein (FAP) antibody mF19 (FIG. 1d) and anti-CD31 antibody (FIG. 1e) to reveal stromal and vascular components, respectively.

A corresponding analysis was performed on a range of human tumors, including breast, lung, colon, kidney and bladder carcinomas and melanoma (Table 1). With the exception of the analyzed non-transitional cell bladder carcinomas, all other tumors revealed expression of EphA3 in the stromal compartment. By immunohistochemistry, some patients with transitional cell carcinoma (TCC) have stromal positivity (data not shown).

TABLE 1

| Tumor Type | Frequency | Stroma | Vasculature |
|---|---|---|---|
| Breast, Infiltrating | 2/6 | + | + |
| Ductal Carinoma | | | |
| Lung | 7/7 | + | + |
| Adenocarcinoma | | | |
| Lung Squamous Cell | 1/1 | + | + |
| Carcinoma | | | |
| Colon | 2/3 | + | + |
| Adenocarcinoma | | | |
| Kidney, renal cell | 3/3 | + | + |
| carcinoma | | | |
| Bladder carcinoma | 6/8 | | + |
| Prostate, | 8/8 | + | +/− |
| Adenocarcinoma | | | |
| Melanoma | 11/15 | + | + |

### Example 2

### EphA3 is Expressed on a Side Population in Human Peripheral Blood Distinct from Circulating Endothelial Progenitor Cells

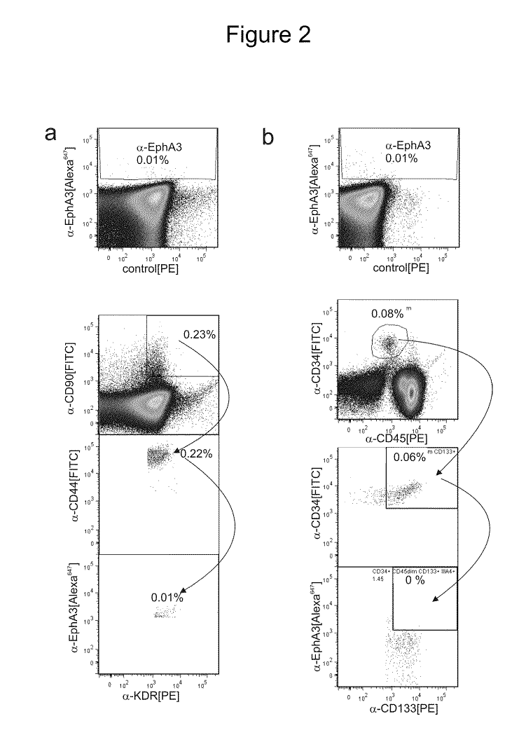

This examples shows that EphA3 is expressed on a population of cells in human peripheral blood that is distinct from circulating endothelial cells. Peripheral blood mononuclear cells were isolated from samples of peripheral blood collected from normal volunteers and cancer patients, using 8 ml BD Vacutainer CPT Tubes as described (Duda, et al., Nature protocols 2:805-810, 2007). Parallel samples from each donor were analyzed (FIG. 2) either for EphA3 alone (top panels) or analyzed by multiparameter flow cytometry for the expression of EphA3 together with the MSC markers CD90, CD44, and KDR (FIG. 2a), or together with expression of endothelial progenitor markers CD34, KDR and CD133 (FIG. 2b). A total of 1×10⁶ cells were analyzed in each of the samples as well as in control samples. The latter included samples containing isotype-matched non-relevant control antibodies, FMO (fluorescence minus one) samples, and unstained samples that were used to determine appropriate detector settings. Interestingly, EphA3⁺ cells were found within the CD90⁺/CD44⁺/KDR⁺ stromal cell population (FIG. 2a), but were not found in the CD45^{dim}/CD34⁺/CD133⁺ CEP cell population (FIG. 2b).

### Example 3

### Flow Cytometric Quantitation of CEP and EphA3⁺ Cells in Peripheral Blood

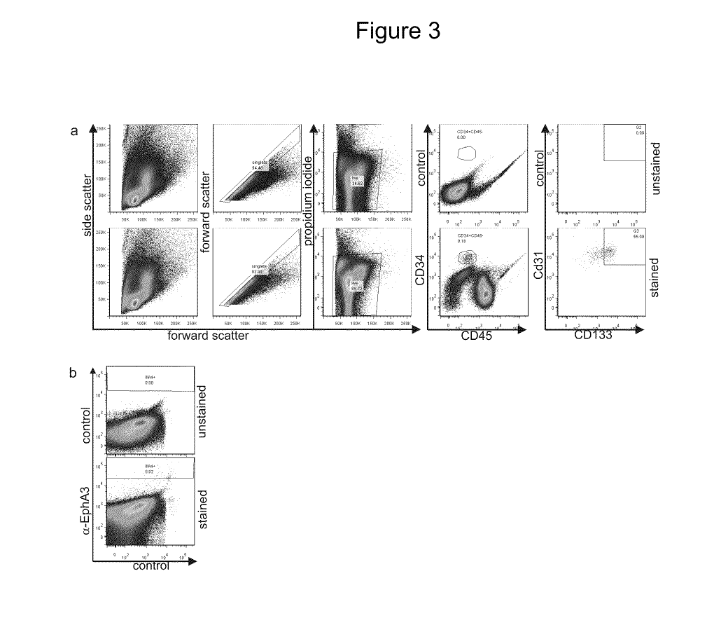

To estimate the concentration of CEPs in blood samples, 1×10⁶ peripheral blood monocytes, which had been incubated with FITC-conjugated anti-CD34, Pacific blue-conjugated anti-CD45, Alexa 674-conjugated anti-CD31 and PE-conjugated anti-CD133, were analyzed sequentially by flow cytometry. The analyzed cells were gated to specifically analyze single cell populations, and control samples outlined above were included to confirm the specificity of the signals (FIG. 3a). Single cells populations from parallel samples were analyzed with α-EphA3 mAb IIIA4, after adjusting the gate for viable single cells populations (FIG. 3b).

### Example 4

### Concentration of CEP and EphA3+ Cells in Peripheral Blood in Cancer Cells

This example shows that the concentration of CEPs and of EphA3⁺ mural (non-endothelial vascular) cells is significantly increased in peripheral blood of cancer patients.

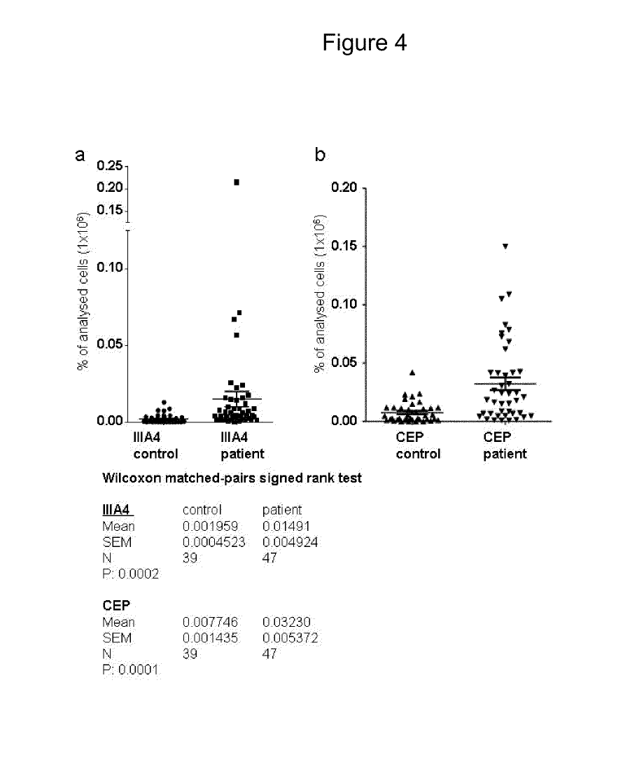

Human circulating mononuclear cells were analyzed for the concentration of EphA3-positive cells (FIG. 4a) using mab IIIA4 or of CEP's (FIG. 4b) using the protocols outlined in FIG. 3. The plot summarizes data from 47 cancer patients and 37 normal volunteers, whereby statistical analysis of the two sample populations was done using the Wilcoxon matched-pairs signed rank test. Analysis of the flow cytometric analysis revealed that both, the number of EphA3-positive cells and of CEP's was significantly higher in the peripheral blood of cancer patients than in normal volunteers.

### Example 5

### Concentration of CEP's and EphA3-Expressing Cells Following Anti-Vascular Therapy

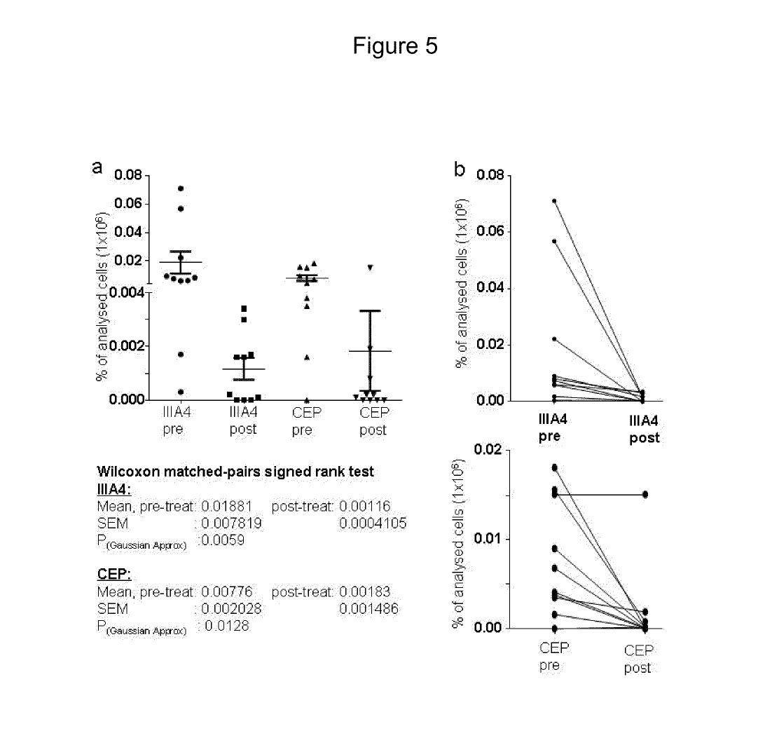

The concentrations of CEP's and EphA3-expressing cells are reduced after anti-vascular therapy. Peripheral blood samples from a total of n=10 colon cancer patients undergoing anti-vascular therapy (Avastin) were analyzed for the presence of EphA3⁺ cells using mab IIIA4 and of CEP's before treatment and 6 weeks later when treatment was terminated. Statistical analysis (Wilcoxon matched-pairs signed rank test) revealed a significant drop in the concentration of EphA3-positive cells and of CEPs in the blood of these patients after treatment (FIG. 5).

In summary, these examples indicate that EphA3 marks a subpopulation of circulating CD34−/CD45−/CD44+/CD90+/KDR+/EphA3+ MSCs, which are phenotypically distinct to CEPs. Similar to the disease-correlated increase in the concentration of CEP's14, these EphA3⁺ MSCs were elevated in the peripheral blood of cancer patients. Anti-vascular treatment reduced the number of EphA3-positive cells in all tested cancer patients to the level found in normal volunteers.

### Example 6

### Use of Antibodies Other than mAb IIIA4 to Detect EphA3 Expression

Mice immunized with recombinant EphA3-Fc fusion protein were used to generate a panel of murine monoclonal antibodies to the extracellular domain of human EphA3 by fusion of spleen B-cells to SP2/0 cells. Hybridoma supernatants were screened for antibodies binding to recombinant human EphA3 extracellular domain and for competition for binding to the site on EphA3 recognized by IIIA4. Four monoclonal antibodies, designated SL-2, SL-5, SL-6 and SL-7 were identified that bind specifically to EphA3 and do not compete for binding to the IIIA4 epitope. Each of the antibodies is a murine IgG1 isotype antibody.

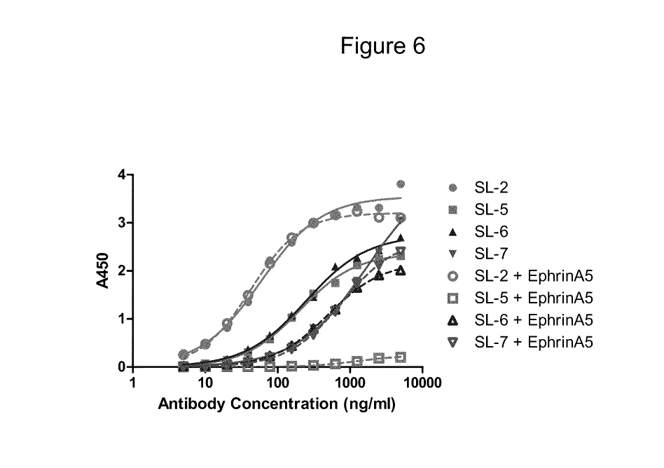

Binding of antibodies to recombinant EphA3 extracellular domain was determined by ELISA using EphA3-Fc in the presence or absence of recombinant extracellular domain of the major ligand for EphA3, EphrinA5. The data shown in FIG. 6 indicate that three of the antibodies (SL-2, SL-6 and SL-7) have equivalent binding activity for EphA3 and EphA3 pre-bound to EphrinA5. The binding of antibody SL-5 is inhibited by the presence of EphrinA5 and therefore SL-5 recognizes an epitope overlapping the Ephrin-binding site. Thus, SL-2, SL-6 and SL-7 are specific for EphA3 but do not bind the Ephrin-binding site or the IIIA4 epitope. This panel of antibodies identifies at least three non-overlapping epitopes on the EphA3 extracellular domain.

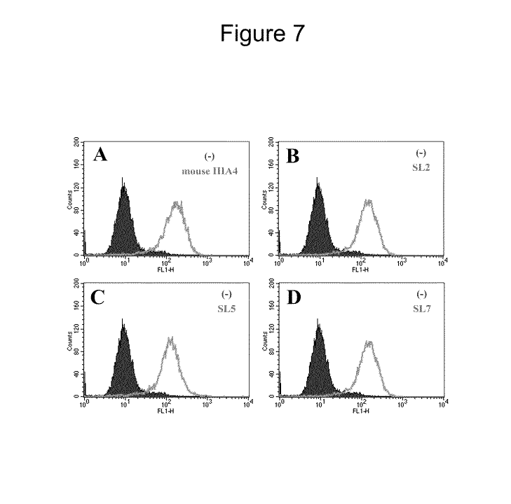

The binding of three of the antibodies, SL-2, SL-5 and SL-7 to native EphA3 on the surface of human LK63 cells was determined by flow cytometry (FIG. 7). LK63 cells were maintained in RPMI 1640 medium supplemented with 10% heat-inactivated fetal bovine serum. For flow cytometry analysis, cells were harvested by centrifugation at 200 g for 5 minutes. Cells were washed once in PBS (Invitrogen) and blocked with 2% bovine serum albumin (BSA)+10 μg/ml rat IgG in PBS for 25 minutes on ice. Cells were incubated with 25 nM purified antibodies for 30 minutes on ice, washed once in cold PBS+0.5% BSA, and incubated in a 1:100 dilution of FITC-conjugated anti-mouse secondary antibody (Jackson Immunoresearch) for 20 minutes on ice. Cells were washed once in cold PBS+0.5% BSA before being re-suspended in 0.5 ml cold PBS for FACS analysis. Propidium iodide (1 μl/sample, Sigma) was used to exclude non-viable cells from FITC channel readings. Cells were analyzed with a FACS Caliber instrument (Beckton Dickinson).

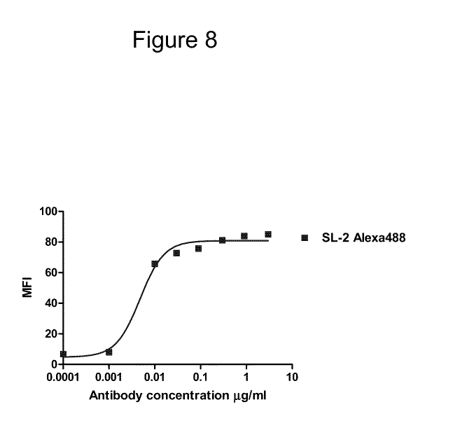

SL-2 antibody was conjugated to Alexa488 by standard methods according to the manufacturer's recommendations. This preparation of antibody has approximately 10 Alexa488 molecules per antibody molecule. The conjugated antibody was used to determine binding of SL-2 to bone marrow cells from a patient diagnosed with acute myeloid leukemia (AML). The data shown in FIG. 8 show that SL-2 provides a sensitive detection reagent for the analysis of EphA3 on primary cells from cancer patients. Maximal binding of antibody was achieved at a concentration of 0.1 μg/ml SL-2.

Each of the antibodies binds to human cells expressing EphA3. Thus, these antibodies are suitable reagents for use in detection of EphA3-expressing cells obtained from biological fluids and patient samples.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications, patent applications, accession numbers, and other references cited in this specification are herein incorporated by reference as if each individual publication or patent application were specifically and individually indicated to be incorporated by reference.

What is claimed is:

1. A method of identifying a patient that has a solid tumor, the method comprising:
providing a sample comprising peripheral blood mononuclear cells from a patient that may have a solid tumor;
detecting a level of EphA3⁺ non-hematopoietic, non-tumor cells in the sample above normal, thereby identifying a patient that has a solid tumor.

2. The method of claim 1, further comprising determining whether the EphA3⁺ cells are CD34⁻.

3. The method of claim 1, further comprising determining whether the EphA3⁺ cells are CD45⁻.

4. The method of claim 1, further comprising determining whether the EphA3⁺ cells express CD44, CD90, and/or KDR.

5. The method of claim 1, wherein the step of detecting the level of EphA3+ non-hematopoietic, non-tumor cells comprises detecting EphA3 expression on the surface of the cells.

6. The method of claim 5, wherein EphA3 expression on the surface of the cells is detected by flow cytometry.

7. The method of claim 5, wherein the step of detecting expression on the surface of the cells comprises contacting the cells with a first antibody that selectively binds to EphA3.

8. The method of claim 7, further comprising contacting the cells with a second antibody that selectively binds to EphA3 at an epitope that is different than the epitope to which the first antibody binds.

9. The method of claim 8, wherein the second antibody is labeled with the same detectable label as the first antibody.

10. The method of claim 1, wherein the patient has a breast carcinoma, a lung adenocarcinoma, a lung squamous cell carcinoma, a colon adenocarcinoma, a renal cell carcinoma, a transitional cell carcinoma, a prostate adenocarcinoma, or a melanoma.

11. The method of claim 1, wherein the step of detecting the level of EphA3+ non-hematopoietic, non-tumor cells comprises an RT-PCR reaction.

12. The method of claim 1, further comprising administering a cancer therapeutic agent to the patient.

13. The method of claim 12, wherein the cancer therapeutic agent is an anti-vascular therapeutic agent

14. The method of claim 13, wherein the anti-vascular therapeutic agent is a vascular endothelial growth factor (VEGF) antagonist or an antibody that activates EphA3.

15. The method of claim 12, wherein the cancer therapeutic agent in an antibody that selectively binds EphA3.

16. A method of monitoring efficacy of a cancer therapeutic agent, the method comprising:
determining the level of EphA3+ non-hematopoietic, non-tumor cells in peripheral blood from a patient that has a solid tumor following a treatment with the cancer therapeutic agent;
comparing the level of EphA3+ non-hematopoietic, non-tumor cells in peripheral blood to the level prior to the treatment with the cancer therapeutic agent.

17. The method of claim 16, wherein the cancer therapeutic agent is an anti-vascular-therapeutic agent.

18. The method of claim 17, wherein the anti-vascular therapeutic agent is a VEGF antagonist or an antibody that activates EphA3.

19. The method of claim 16, wherein the cancer therapeutic agent is an antibody that selectively binds EphA3⁺.

20. The method of claim 16, further comprising determining whether the EphA3⁺ cells are CD34⁻.

21. The method of claim 16, further comprising determining whether the EphA3⁺ cells are CD45⁻.

22. The method of claim 16, further comprising determining whether the EphA3⁺ cell express CD44, CD90, and/or KDR.

23. The method of claim 16, wherein the step of determining the level of EphA3⁺ non-hematopoietic, non-tumor cells comprises detecting EphA3 expression on the surface of the cells

24. The method of claim 23, wherein EphA3 expression on the surface of the cells is detected by flow cytometry.

25. The method of claim 23, wherein the step of detecting expression on the surface of the non-hematopoietic cells comprises contacting the cells with a first antibody that selectively binds to EphA3

26. The method of claim 25, further comprising contacting the cells with a second antibody that selectively binds to a different EphA3 epitope.

27. The method of claim 26, wherein the first and the second antibody are labeled with the same detectable label.

28. The method of claim 16, wherein the cancer therapeutic agent is a therapeutic antibody that selectively binds to EphA3 and the step of determining the level of EphA3+ non-hematopoietic, non-tumor cells comprises contacting the cells with an antibody that binds to an epitope different to the epitope to which the therapeutic antibody binds.

29. The method of claim 16, wherein the patient has a breast carcinoma, a lung adenocarcinoma, a lung squamous cell carcinoma, a colon adenocarcinoma, a renal cell carcinoma, a transitional cell carcinoma, a prostate adenocarcinoma, or a melanoma.

30. The method of claim 16, wherein the step of detecting expression of EphA3+ comprises an RT-PCR reaction.

31. A kit for detecting the presence of non-hematopoietic cells in a sample, the kit comprising a first antibody that selectively binds to an EphA3 epitope and a second antibody that selectively binds to a different EphA3 epitope.

32. The kit of claim 31, wherein the first and the second antibodies are labeled with the same detectable label.

33. The kit of claim 31, further comprising an antibody that binds to a surface marker selected from the group consisting of CD34, CD45, CD90, and KDR.

34. A method of identifying the presence or absence of a non-hematopoietic, non-tumor population of EphA3⁺ cells in peripheral blood from a patient, the method comprising:
providing a sample comprising peripheral blood cells from the patient;
detecting the presence or absence of expression of EphA3⁺ in non-hematopoietic, non-tumor cells in the sample, wherein the presence of EphA3+ expression on non-hematopoietic, non-tumor cells is indicative of the presence of a solid tumor.
